(19) United States
(12) Patent Application Publication
(10) Pub. No.: US 2010/0211146 A1
(43) Pub. Date: Aug. 19, 2010

(54) PHOTOELECTRIC ACTIVATION OF NEURONS USING NANOSTRUCTURED SEMICONDUCTORS

(76) Inventors: BEN W. STROWBRIDGE, Cleveland Heights, OH (US); CLEMENS BURDA, Cleveland Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/706,107

(22) Filed: Feb. 16, 2010

Related U.S. Application Data
(60) Provisional application No. 61/152,324, filed on Feb. 13, 2009.

(51) Int. Cl. A61N 1/36

(52) U.S. Cl. 607/116; 977/904

(57) ABSTRACT

A photoelectric stimulating electrode for photoelectric activation of a neuron includes a plurality of semiconductor nanoparticles adapted to be positioned proximate a neuron. The semiconductor nanoparticles upon excitation with light of a first wave length generate an electric field and/or current effective to stimulate the neuron.

### RELATED APPLICATION

This application claims priority from U.S. Provisional Application No. 61/152,324, filed Feb. 13, 2009, the subject matter, which is incorporated herein by reference.

### GOVERNMENT FUNDING

This invention was made with government support under Grant Nos. R01-DC04285 and R01-NS33590 awarded by The National Institutes of Health. The United States Government has certain rights in the invention.

### FIELD OF THE INVENTION

The present invention relates to a device and method for stimulating cells and particularly relates to the photoelectric activation of neurons in brain tissue using nanostructured semiconductors.

### BACKGROUND

In various medical fields, the use of artificial stimulation devices, or prosthesis, to stimulate damaged cells and/or tissue, which are no longer responsive to natural stimuli is well known. These devices mimic natural impulses and act to re-establish the natural stimulation path.

One of the best examples of the success of such an approach is the use of the cochlear implant to restore partial hearing in profoundly deaf people. A person is diagnosed as profoundly deaf if either a very large number of hair cells or auditory neurons throughout the cochlea, the spiral-shaped cavity of the inner ear, are damaged. Cochlear implants use electrical stimulation to directly excite the remaining auditory neurons, which connect the ear to the brain.

Applications of electrical stimulation systems are not limited to cochlear implants. They include brain neuro-stimulation (pain relief, tremor control, treatment of cerebral palsy, treatment of Parkinson's disease, visual cortex implants for the blind), spinal neuro-stimulation (pain relief, peripheral vascular flow enhancement), peripheral nerve stimulation (pain relief, phrenic nerve pacing), retinal implants, heart pacemakers, tissue-growth stimulation and inhibition, etc.

Functional Electrical Stimulation (FES) is used to produce, by means of electrical stimulation, contractions in muscles either injured or paralyzed due to central nervous system lesions. In the case of FES, arrays of electrodes are implanted under the skin and used to choreograph movement in the patient's muscles.

Applications for this approach are found, for example, in cases of stroke, spinal cord injury, head injury, cerebral palsy, and multiple sclerosis. Here, too, resolution is limited by the size of the wires used for electrical stimulation.

Efforts are underway to develop visual prostheses, both retinal and cortical. Retinal prostheses aim to restore some form of vision to patients that are blind owing to a degenerative condition, such as retinitis pigmentosa or age-related macular degeneration, by bypassing the photoreceptor cells of the retina, which have become dysfunctional and electrically stimulating the relatively intact retinal ganglion cells which connect the eye to the visual cortex of the brain. Electrical stimulation of the retinal ganglion cells creates the sensation of a spot of light (or phosphene) in the spatial vicinity of the stimulation. Cortical prostheses may be used to treat patients with secondary blindness not due to retinal or optic nerve disease. The difficulty with cortical implants lies in the need for intracranial surgery and the complexity of brain geometry. Nevertheless, both types of prostheses are faced with the problems inherent with electrical stimulation: injury incurred by neurons under chronic use and lack of specificity. U.S. Pat. No. 6,458,157 discloses an apparatus in which all tissue-contacting components may be fabricated from materials known to be well tolerated by human tissue. While the '157 patent discloses attempts that have been made to limit injury due to long-term use, the matter of specificity is not expressly addressed.

In general, traditional methods and devices for direct electrical neuro-stimulation lack spatial, physiological and strength specificities. Furthermore, they are prone to electrical interference from the environment. For example, electrical stimulation of the visual cortex produces phosphenes (or blurred) spots rather than pixel-like (or well-defined) spots. Stimulating tactile sense through electrical stimulation of specific neuronal cells is practically impossible without stimulating muscles and/or a temperature response, producing hitching or pain. A stimulation device permitting stimulation of specific neural ganglion cells would allow for better control of the stimulation process.

Recent developments in nanotechnology (nanoshells, quantum dots (QDs), micelles), photodynamic therapy and photo-imaging offer new possibilities for improving specificity. These new technologies provide ways to cage, tag, and locate molecules thus allowing the regulation and monitoring of optical stimulation mechanisms. Of particular interest are molecular structures or compounds that undergo changes in their properties (chemical affinity, conformal structure or composition) upon exposure to light (photoactivated changes). Following photoactivation, these molecules can react with other molecules or cells or emit light. In some cases, molecules undergo photoactivation only in the presence of certain other molecules or cells thus allowing these photoactivated molecules to be used as targets for locating, monitoring, imaging or destroying these other molecules or cells when lighted. For example, U.S. Pat. No. 6,668,190 (IEZZI et al.) discloses a drug delivery system that includes a fluid channel for delivering a drug to one of a number of sites and a light channel for delivering light to an area near one of the sites for photoactivating caged and/or non-caged molecules of the drug to stimulate neurological tissue.

### SUMMARY OF THE INVENTION

The present invention relates to a photoelectric stimulating electrode for photoelectric activation of a neuron. The electrode includes a plurality of semiconductor nanoparticles adapted to be positioned proximate the neuron. The semiconductor nanoparticles upon excitation with light of a first wavelength can generate an electric field and/or current effective to stimulate the neuron.

In an aspect of the invention, the semiconductor nanoparticles can generate an electric field and/or current effective to stimulate the neuron upon excitation with visible and/or near-infrared light. The visible and/or infrared light can have, for example, a wavelength of about 650 nm to about 2200 nm.

In another aspect of the invention, the electrode can include a biocompatible substrate and the semiconductor nanoparticles can be provided in and/or on the biocompatible substrate. The substrate can be substantially transparent to light effective to excite the semiconductor nanoparticles. The substrate can also have an area that is adapted to be positioned proximate a plurality of neurons, and the semiconductor nanoparticles can be provided in an array within the area. The semiconductor nanoparticles provided in the array can be selectively excited by light to selectively activate individual neurons proximate the substrate.

In a further aspect of the invention, the semiconductor nanoparticles can include a semiconductor material having a band gap less than about 2 eV at a temperature of 300K. Examples of semiconductor materials having a band gap less than about 2 eV are PbSe and Si.

The present invention also relates to an apparatus for photoelectric activation of a neuron. The apparatus includes a plurality semiconductor nanoparticles adapted to be positioned proximate the neuron. The semiconductor nanoparticles can generate an electric field and/or current effective to stimulate the neuron upon excitation with light of a first wavelength. The apparatus also includes a light generating means for generating light having the first wavelength effective to excite the semiconductor nanoparticles to generate the electric field and/or current.

The present invention further relates to a method of stimulating a neuron. The method includes positioning a plurality of semiconductor nanoparticles proximate the neuron. The semiconductor nanoparticles can generate an electric field and/or current effective to stimulate the neuron upon excitation with light of a first wavelength. The plurality of semiconductor nanoparticles are then excited with light having the first wavelength to generate the electric field and/or current. The neurons can tissue of the central nervous or peripheral nervous and be in vivo or ex vivo.

### BRIEF DESCRIPTION OF THE DRAWINGS

FIG. 1 illustrates a schematic drawing of a nanostructured photoelectric stimulating electrode in accordance with an aspect of the invention.

FIG. 2 illustrates a schematic diagram (a) and video micrograph (b) of a neuron recorded through a recording electrode (Rec) contacting a L2/3 pyramidal cell (PC) and nanostructured photoelectric stimulating electrode (PE Stim). c) Schematic description of the process for coating the outside (left) and inside (right) surfaces of closed-head glass microtips with nanoparticle thin films through chemical bath deposition (CBD) as confirmed by SEM.

FIG. 3 illustrates a) XRD of the thin film of PbSe nanoparticles coated on a glass substrate (the bars below the signals correspond to the standard JCPDS standards PbSe. b-d) SEM images of the PbSe nanoparticle film coated on the outside of the glass microtip: b), c) side view, d) enlargement showing the PbSe nanoparticles.

FIG. 4 illustrates a) photopotential recorded from the photoexcited stimulation photoelectrode (PE Stim) using two recording microelectrodes (Rec) placed near the photoelectrodes. Traces are offset for clarity. b) The schematic setup for photoinduced electrical field measurement.

FIG. 5 illustrates a) comparison of photoelectric activation (top; 830 nm pulsed beam) and depolarization of the same L2/3 pyramidal cell with by direct-current injection (bottom); b) Comparison of rapid modulation of photoelectric excitation of mitral cells (MC) using sinusoidal light-intensity waveforms (top panel) with a direct-current injection step (bottom panel). The holding potential (in mV) is indicated above each trace. The top panel shows sub- and suprathreshold responses to 10 and 18 mW 5 Hz sine wave photoelectric stimuli, respectively; troughs between cycles correspond to 0 mW excitation. Action potentials truncated. The bottom panel illustrates the response of the same mitral cell to 10 mW modulated photoelectric excitation in the presence of 1 μm tetrodotoxin (TTX). c) Response of the same neuron to 5 Hz sinusoidal current injection. Responses to both photoelectric (b) and current injection (c) show a similar phase lag (arrow and dotted vertical line).

### DETAILED DESCRIPTION

The present invention relates to an apparatus and method for photoelectric modulation of a neuron. The apparatus can be used to modulate neuron activation and particularly neuron activation in a subject as well as treat a subject with neural disorder or medical condition.

As used herein, the terms “modulate” or “modulating” can refer to causing a change in neuronal activity, chemistry and/or metabolism. The change can refer to an increase, decrease, or even a change in a pattern of neuronal activity. The terms may refer to either excitatory or inhibitory stimulation, or a combination thereof. The terms can also be used to refer to a masking, altering, overriding, or restoring of neuronal activity.

As used herein, the term “subject” can refer to any warm-blooded organism including, but not limited to, human beings, pigs, rats, mice, dogs, goats, sheep, horses, monkeys, apes, rabbits, cattle, etc.

As used herein, the terms “treat” or “treating” shall have their plain and ordinary meaning to one skilled in the art of pharmaceutical or medical sciences. For example, “treat” or “treating” can mean to prevent or reduce a pain in a subject.

In an aspect of the present invention, the apparatus includes a photoelectric stimulating electrode. The photoelectric stimulating electrode can generate an electric field and/or current effective to stimulate a neuron upon excitation with light of a first wavelength. The apparatus also includes a light generating mean for generating light having the first wavelength effective to excite the photoelectric stimulating electrode and stimulate the neuron.

The photoelectric electrode can include a plurality of photoelectric semiconductor nanoparticles that can be excited with a wavelength of light resulting in detectable generation of an electric field, voltage, or current effective to stimulate the neuron. The photoelectric semiconductor nanoparticles can be formed from aggregates of anywhere from a few hundred to tens of thousands of semiconductor atoms. The semiconductor nanoparticles can have an average diameter ranging from about 3 nm to about 100 nm. The photoelectric semiconductor nanoparticle can also be biocompatible with and/or substantially non-toxic to living tissue and neural cells when positioned proximate to the cells or tissue.

With respect to shape, the semiconductor nanoparticles can include elongated particle shapes, such as nanowires, or irregular shapes, in addition to more regular shapes, such as spherical, hexagonal, and cubic nanoparticles, and mixtures thereof. Additionally, the nanoparticles may be single-crystalline, polycrystalline, or amorphous in nature. As such, a variety of types of semiconductor nanoparticle materials may be created by varying the attributes of composition, size, shape, and crystallinity of semiconductor nanoparticles. Examples include single or mixed elemental composition (including alloys, core/shell structures, doped nanoparticles, and combinations thereof) single or mixed shapes and sizes (and combinations thereof), and single form of crystallinity (or a range or mixture of crystallinities, and combinations thereof).

In an aspect of the invention, the semiconductor nanoparticles can be formed of various semiconductor materials, such as Group II-VI semiconductor elements of the periodic table that can be readily formed into semiconductor nanoparticles. Examples of semiconductor materials that can be used to form the semiconductor nanoparticles include MgS, MgSe, MgTe, CaS, CaSe, CaTe, SrS, SrTe, BaS, BaTe, ZnS, ZnSe, ZnTe, CdS, CdSe, CdTe, GaAs, GaP, GaSb, InGaAs, InP, InN, InSb, InAs, AlP, AlSb, AlS, PbS, PbSe, Ge, Si, and mixtures thereof.

In another aspect of the invention, the semiconductor nanoparticles can be formed from semiconductor materials that can be readily excited by light having a wavelength in the visible to near infrared range (e.g., about 650 nm to about 2200 nm) to generate an electric field and/or current effective to stimulate or activate a neuron to which the semiconductor nanoparticles are positioned proximate (e.g., directly abutting to about 20 μm). The semiconductor nanoparticles can also be formed from semiconductor materials having a narrow band gap. By narrow band gap, it is meant semiconductor materials having a band gap less than about 2 eV at a temperature of 300K. Examples of semiconductor materials having a band gap less than about 2 eV are PbSe and Si.

One skilled in the art will appreciate that a variety of techniques can be employed to form semiconductor nanoparticles for use in accordance with the present invention. For example, techniques of forming semiconductor nanoparticles are disclosed, for example, in Zhao, et al., Angew Chem Int Ed Engl. 2009; 48(13):2407-10, Qiu et al., Angew. Chem. 2006, 118, 5784-5787, and Sarkar et al., Chem. Mater. 2007, 19, 879-888 as well as U.S. Patent Application Publication Nos. 2007/0175507, 2009/0224216, 2009/0239330, and 2009/0293659, all of which are incorporated by reference in their entirety.

In another aspect of the present invention, the photoelectric semiconductor nanoparticles can be provided as a thin film (e.g., about 1 μm to about 100 μm). The film of the semiconductor nanoparticles can be deposited on/or in a biocompatible substrate that can be positioned proximate a neuron to be stimulated. In one example, the substrate can include a micro-scale or nano-scale ceramic, glass, plastic, or polymer based substrate. The substrate can be substantially transparent (e.g., at least 50% transparent) to the wavelength of light used to excite the semiconductor nanoparticles.

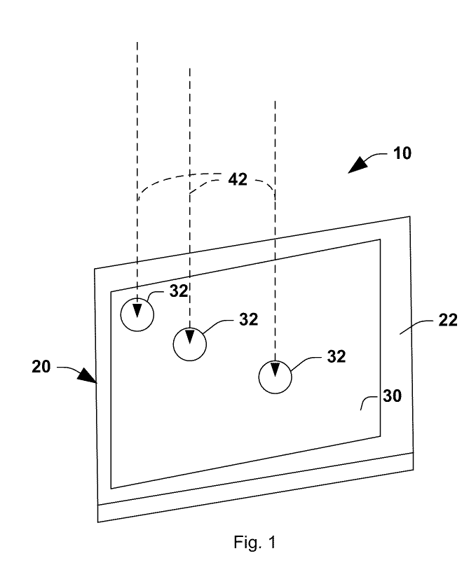

FIG. 1 is a schematic illustration of a photoelectric stimulating electrode in accordance with an aspect of the invention. The electrode 10 includes a substrate 20 with a surface 22 that defines an area of the substrate 20 to be positioned proximate a plurality of neurons in a subject. A film of semiconductor nanoparticles 30 (e.g., PbSe or Si) can be deposited in an array on the area of the surface 22 of the substrate 20. The semiconductor nanoparticles 30 or regions 32 of semiconductor nanoparticles provided in the array can be selective excited by light 42 to selectively activate individual neurons (not shown) proximate the substrate 10. Selective activation of neurons can allow for spatial control of regions of central nervous system tissue, such as brain tissue, that have not been previously achieved using conventional electrodes.

The light generating means used to stimulate the photoelectric semiconductor nanoparticles may include a single monochromatic light source, such as a light-emitting diode or laser diode, or a number of such sources. Light from the light generating means can be used to photoactivate the photoelectric semiconductor nanoparticles, which then directly or indirectly stimulate specific neurons, neural tissue, or nervous system functions. The wavelength of the light is chosen to match the photoactivation wavelength of the semiconductor nanoparticles.

In an aspect of the invention, the wavelength of light used to excite the semiconductor nanoparticles can include with visible and/or near-infrared light, such as visible and/or infrared light having a wavelength of about 650 nm to about 2200 nm. Advantageous, the light used to excite the semiconductor nanoparticles can include having a wavelength in near infrared range. Light in the near infrared range can more readily pass through tissue of a subject to excited the semiconductor nanoparticles and does not stimulate fluorophores in the subject.

In one example, where the semiconductor particles are in the form of a thin film that comprises a plurality of PbSe semiconductor nanoparticles provided, the light generating means can include a near infrared (NIR) light source. In an aspect of the invention, the NIR light source can generate a light signal at a wavelength of about 830 nm, which is effective to excite the PbSe nanoparticles and generate an electric field or current to modulate the neuron.

A neural cell can be stimulated with the semiconductor nanoparticles by placing and/or positioning the semiconductor nanoparticles in the vicinity proximate the neural cells to be stimulated. In one example, the semiconductor nanoparticles can be positioned proximate the neural by placing a surface of a substrate (e.g., glass micropipette) coated with a thin film of the semiconductor nanoparticles adjacent the neural cells. In another example, the semiconductor nanoparticles can be provided in a biocompatible and/or photoconductive polymer and then locally administered to the neuron being stimulated by, for example, direct injection.

Upon positioning of the semiconductor nanoparticles proximate the neural cell, the semiconductor nanoparticles can be excited with the appropriate wavelength of light from the light generating means to generate an electric field and/or current. The light from the light generating means can be transmitted directly to the semiconductor nanoparticles or though a transmission means, such as the substrate on which the semiconductor nanoparticles can be potentially placed.

The photoelectric stimulation of the neurons can be episodic, continuous, phasic, in clusters, intermittent, upon demand by the subject or medical personnel, or pre-programmed to respond to a sensor (not shown) (e.g., a closed-loop system). Additionally, photoelectric stimulation may be applied to the neuron simultaneously or sequentially.

The photoelectric stimulating apparatus can be provided in and/or on a subject to treat a neural injury or medical condition by neuromodulating and/or neurostimulating neural cells of the subject. In the context of the present invention, the term “medical condition” can refer to any movement disorders, epilepsy, cerebrovascular diseases, autoimmune diseases, sleep disorders, autonomic disorders, urinary bladder disorders, abnormal metabolic states, disorders of the muscular system, infectious and parasitic diseases neoplasms, endocrine diseases, nutritional and metabolic diseases, immunological diseases, diseases of the blood and blood-forming organs, mental disorders, diseases of the nervous system, diseases of the sense organs, diseases of the circulatory system, diseases of the respiratory system, diseases of the digestive system, diseases of the genitourinary system, diseases of the skin and subcutaneous tissue, diseases of the musculoskeletal system and connective tissue, congenital anomalies, certain conditions originating in the perinatal period, and symptoms, signs, and ill-defined conditions.

Pain treatable by the present invention can be caused by conditions including, but not limited to, migraine headaches, including migraine headaches with aura, migraine headaches without aura, menstrual migraines, migraine variants, atypical migraines, complicated migraines, hemiplegic migraines, transformed migraines, and chronic daily migraines, episodic tension headaches, chronic tension headaches, analgesic rebound headaches, episodic cluster headaches, chronic cluster headaches, cluster variants, chronic paroxysmal hemicranias, hemicrania continua, post-traumatic headache, post-traumatic neck pain, post-herpetic neuralgia involving the head or face, pain from spine fracture secondary to osteoporosis, arthritis pain in the spine, headache related to cerebrovascular disease and stroke, headache due to vascular disorder, reflex sympathetic dystrophy, cervicalgia (which may be due to various causes, including, but not limited to, muscular, discogenic, or degenerative, including arthritic, posturally related, or metastatic), glossodynia, carotidynia, cricoidynia, otalgia due to middle ear lesion, gastric pain, sciatica, maxillary neuralgia, laryngeal pain, myalgia of neck muscles, trigeminal neuralgia (sometimes also termed tic douloureux), post-lumbar puncture headache, low cerebro-spinal fluid pressure headache, temporomandibular joint disorder, atypical facial pain, ciliary neuralgia, paratrigeminal neuralgia (sometimes also termed Raeder's syndrome); petrosal neuralgia, Eagle's syndrome, idiopathic intracranial hypertension, orofacial pain, myofascial pain syndrome involving the head, neck, and shoulder, chronic migraneous neuralgia, cervical headache, paratrigeminal paralysis, SPG neuralgia (sometimes also termed lower-half headache, lower facial neuralgia syndrome, Sluder's neuralgia, and Sluder's syndrome), carotidynia, vidian neuralgia, causalgia, and/or a combination of the above.

Movement disorders treatable by the present invention may be caused by conditions including, but not limited to, Parkinson's disease, cerebropalsy, dystonia, essential tremor, and hemifacial spasms.

Epilepsy treatable by the present invention may be, for example, generalized or partial.

Cerebrovascular disease treatable by the present invention may be caused by conditions including, but not limited to, aneurysms, strokes, and cerebral hemorrhage.

Autoimmune diseases treatable by the present invention include, but are not limited to, multiple sclerosis.

Sleep disorders treatable by the present invention may be caused by conditions including, but not limited to, sleep apnea and parasomnias.

Autonomic disorders treatable by the present invention may be caused by conditions including, but not limited to, gastrointestinal disorders, including but not limited to gastrointestinal motility disorders, nausea, vomiting, diarrhea, chronic hiccups, gastroesphageal reflux disease, and hypersecretion of gastric acid, autonomic insufficiency; excessive epiphoresis, excessive rhinorrhea; and cardiovascular disorders including, but not limited, to cardiac dysrythmias and arrythmias, hypertension, and carotid sinus disease.

Urinary bladder disorders treatable by the present invention may be caused by conditions including, but not limited to, spastic or flaccid bladder.

Abnormal metabolic states treatable by the present invention may be caused by conditions including, but not limited to, hyperthyroidism or hypothyroidism.

Disorders of the muscular system treatable by the present invention can include, but are not limited to, muscular dystrophy, and spasms of the upper respiratory tract and face.

Neuropsychiatric or mental disorders treatable by the present invention may be caused by conditions including, but not limited to, depression, schizophrenia, bipolar disorder, and obsessive-compulsive disorder.

As used herein, the term “headache” can refer to migraines, tension headaches, cluster headaches, trigeminal neuralgia, secondary headaches, tension-type headaches, chronic and epsisodic headaches, medication overuse/rebound headaches, chronic paroxysmal hemicrinia headaches, hemicranias continua headaches, post-traumatic headaches, post-herpetic headaches, vascular headaches, reflex sympathetic dystrophy-related headaches, crvicalgia headaches, caroidynia headaches, sciatica headaches, trigeminal headaches, occipital headaches, maxillary headaches, diary headaches, paratrigeminal headaches, petrosal headaches, Sluder's headache, vidian headaches, low CSF pressure headaches, TMJ headaches, causalgia headaches, myofascial headaches, all primary headaches (e.g., primary stabbing headache, primary cough headache, primary exertional headache, primary headache associated with sexual activity, hypnic headache, and new daily persistent headache), all trigeminal autonomic cephalagias (e.g., episodic paroxysmal hemicranias, SUNCT, all probable TACs, and SUNA), chronic daily headaches, occipital neuralgia, atypical facial pain, neuropathic trigeminal pain, and miscellaneous-type headaches.

It will be appreciated that any number of disorders and/or conditions in which neuromodulation is desired can be treated with the apparatus of the present invention need not be limited the preceding list. Of course, numerous modifications or combinations of these preferred embodiments could be made to the apparatus above without departing from the scope of the present invention.

### EXAMPLES

### Formation of Nanostructured Semiconductors

Nanostructured PbSe films were deposited on glass microtips according to the procedures outlined in References 15 and 16. First deposition bath solutions were prepared from 0.05 m lead acetate and 1.5 m citric acid in 7.5 mL deionized water; then 2.5 mL of 0.05 m sodium selenosulfate (Na₂SeSO₃ in excess Na₂SO₃) was added (prepared by refluxing 0.2 m Se with 0.5 m Na₂SO₃ for several hours until all Se powder had dissolved). Then the glass microtips were dipped into the bath solutions or the bath solution was injected into the microtip by a capillary; the microtips were then placed into an oven for curing at 80° C. The prepared nanostructured PbSe coated glass microtips were rinsed with distilled water and ethanol and dried under Ar.

The same PbSe nanoparticles were also deposited on a glass substrate under the same conditions by using the same precursor solution, and these nanoparticles were used for the XRD measurement. The glass substrate (22×40 mm), whose thickness is about 0.13-0.16 mm, was purchased from Corning Glass Works (USA) and cut into 11×20 mm pieces with a diamond cutter. Photopotential measurements of the PbSe-nanoparticle-coated photoelectrode were made by placing two tungsten microelectrodes near the photoelectrode and irradiating with visible light from a 75 W Xe lamp introduced by optical fibers under a microscope objective; the photopotentials were recorded on a CH instrument Electrochemical Station 6301B.

### Photoelectric Activation of Brain Slices

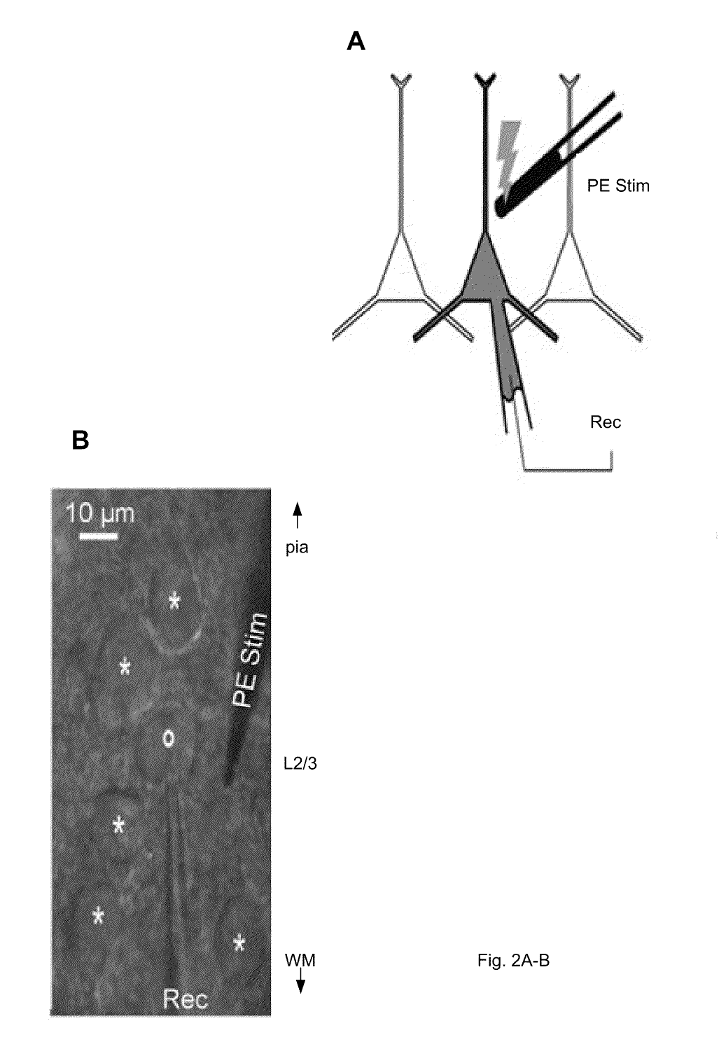

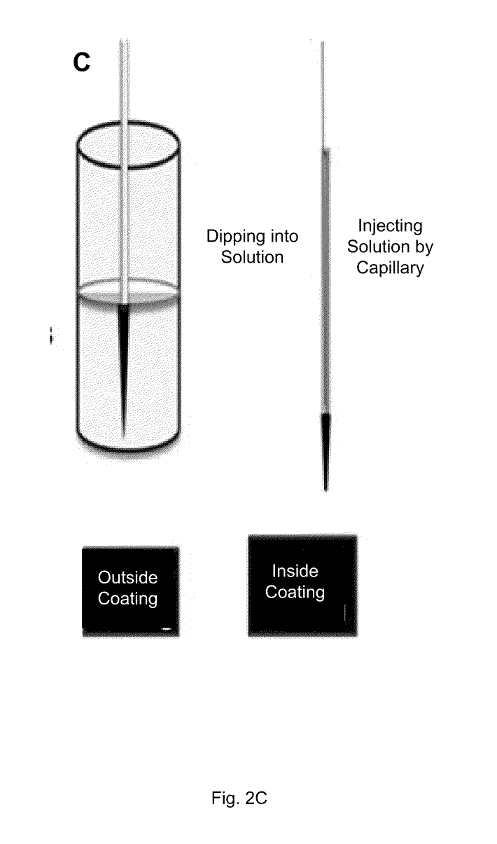

Acute brain slices were prepared from the hippocampi or olfactory bulbs of P14-21 Sprague Dawley rats using standard methods. Slices (300 μm thick) from both brain regions were maintained in a submerged recording chamber at 30° C. and imaged on an upright fixed-stage microscope (Olympus BX51WI) using infrared differential interference contrast (IR-DIC) optics and a frame-transfer video camera (Cohu 6412-2000; FIG. 2b). Slices were superfused with an artificial cerebrospinal fluid containing the following: 124 mm NaCl, 3 mm KCl, 1.23 mm NaH₂PO₄, 1.2 mm MgSO₄, 26 mm NaHCO₃, 10 mm dextrose, and 2.5 mm CaCl₂ equilibrated with 95% O₂/5% CO₂. Whole-cell current-clamp recordings were made using an Axopatch 1D amplifier (Axon Instruments) and pipettes filled with an internal solution that contained: 140 K methylsulfate, 4 mm NaCl, 10 mm HEPES, 0.2 mM EGTA, 4 mm MgATP, 0.3 mm Na₃GTP, and 10 mm phosphocreatine (4-8 MΩ resistance). Electrophysiological records were low-pass-filtered at 2 kHz and digitized at 5 kHz (Instrutech ITC18) using custom Matlab software. A second patch-clamp photoelectrode (PE), which was coated either outside or inside with PbSe nanoparticles, was placed within the same field-of-view of the 60× water-immersion objective as the recording electrode. The nanostructured photo-electrode was positioned near the appropriate cell body (within 10-20 μm) under video microscopy (FIG. 1b) after breakthrough to the whole-cell recording mode. We used a Verdi V10 pump laser and Mira 900 Ti:sapphire oscillator (Coherent) to illuminate the nanoparticles. High-speed XY galvanometer scanners (Cambridge Technology 6210) and custom Visual Basic 6, and Matlab software controlled the beam position within the microscope field-of-view. The laser beam was continuously scanned over the electrode tip in a Lissajous pattern that repeated at 1 kHz; the scan pattern typically covered 20×20 μm. Optical illumination was controlled by varying the control voltage to a Pockels cell intensity modulator (ConOptics) positioned in the laser beam path.

### Results

Using nanoparticle-coated photoelectrodes, we have produced photoelectrical activation patterns in slices of rat hippocampus and olfactory bulb by addressing the photoelectrode (PE) with focused near-infrared (NIR) light signals at λ=830 nm.

Earlier work on the photoelectric excitation of cultured cells used mercury-based thin-film electrodes, in which ensembles of cultured neurons grown on the thin films were activated through photoinduced currents. In the examples, a light-activatable nanoparticle thin film was coated on the inside surface of a pulled-glass microtip, which eliminated any observable neural toxicity during the experiment. With these photoelectrodes, no molecular biology manipulations were necessary, and no direct contact of the neurons with nanoparticle-coated surfaces was required (with the inside-coated tips). Unlike conventional stimulating electrodes, this method required no wiring or electrical power and instead relied only on infrared light to stimulate synaptic inputs to neural circuits. Neurons in brain slices were directly stimulated. In addition to using photoelectric stimuli to define specific neural processing steps in brain slices, the same technology will likely prove useful clinically to activate brain regions and damaged nerves. The schematic diagram and video micrograph of the photoelectrical activation of neurons in a brain slice is shown in FIG. 2.

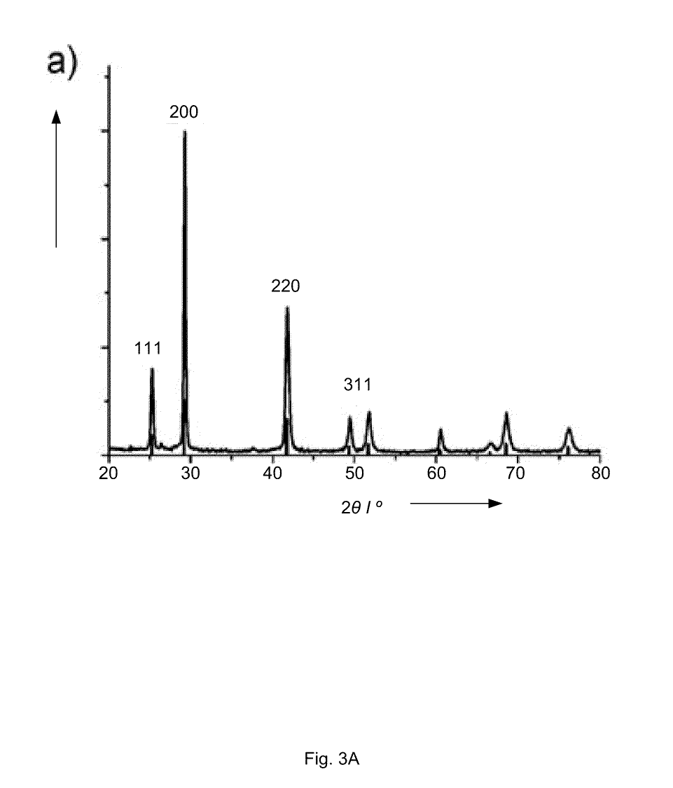

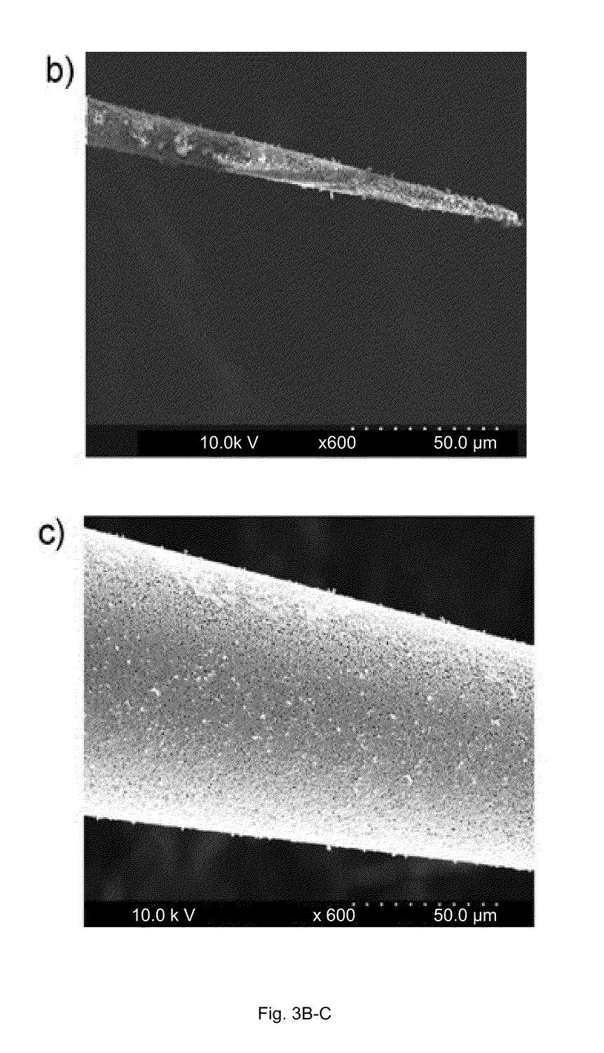

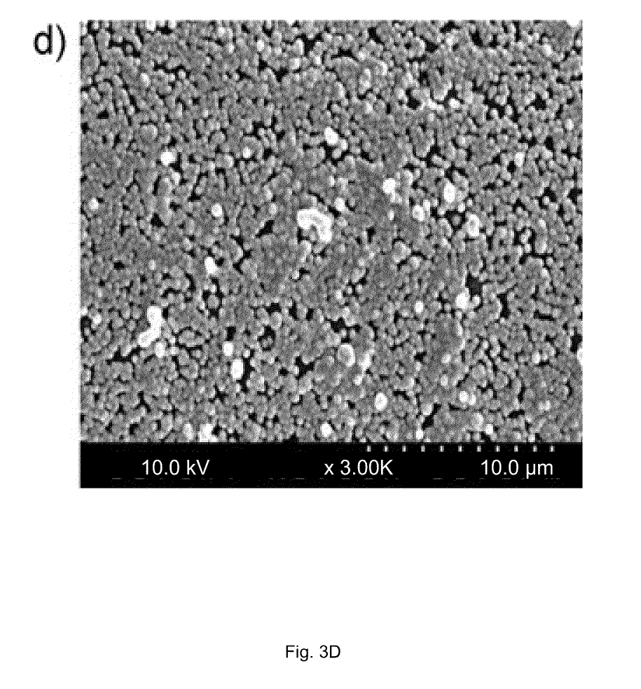

PbSe is a Group IV-Group VI semiconductor with a narrow bulk band-gap energy of 0.26 eV and is commonly used for IR photodetectors. The film's composition and structure were characterized by scanning electron microscopy (SEM) and X-ray diffraction (XRD), respectively. XRD revealed a rock salt structure known for bulk PbSe (FIG. 3a). Scherrer analysis of the XRD provided an estimate of the grain sizes of approximately 100 nm as confirmed by the SEM; this corresponds to an absorption onset well beyond the excitation wavelengths used in this study. The SEM images of the photoelectrodes (FIG. 3b-d) showed that a uniform thin film of PbSe nanoparticles was coated on the outside surface of the glass microtips. Earlier measurements showed that in PbSe films the majority charge carriers are holes, leading to intrinsic p-type nanostructures.

PbSe absorbed broadly in the visible to infrared spectral range, and it is convenient to use 830 nm light from a mode-locked Ti:sapphire laser for photoexcitation since these experiments can also involve two-photon visualization of the neurons filled with Alexa or Ca²⁺-sensitive dyes. Because of the low excitation energy of PbSe, a one-photon excitation process at conditions of low photon flux was sufficient to trigger the neuron response. We developed both outside- and inside-coated microtip pipettes (FIG. 3), principally for defining the physical and biological properties of the photoinduced effects, such as absorption, neural triggering, and phototoxicity, since we can position them close (about 10 μm) to defined neuronal components like cell bodies and dendrites.

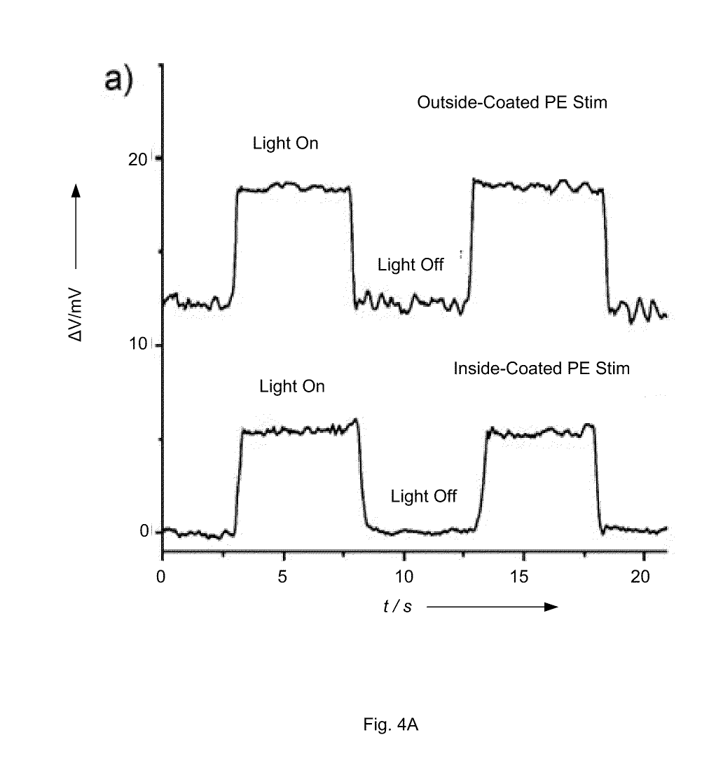

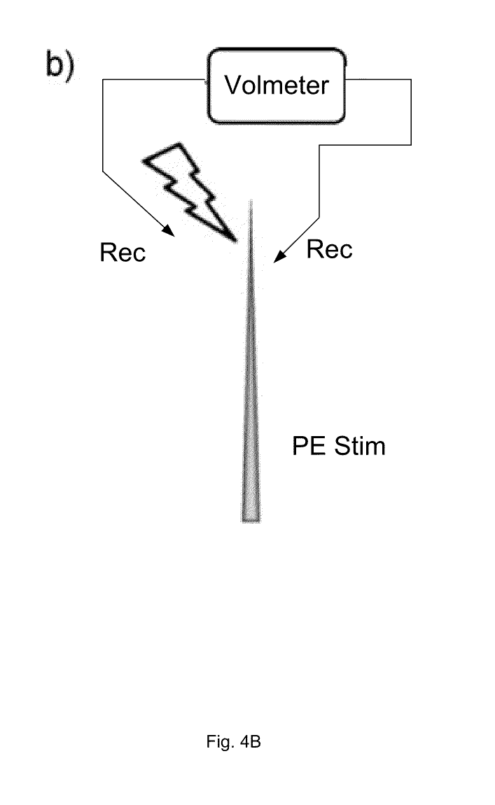

Outside-coated photoelectrodes with exposed PbSe nanoparticle films did not show any noticeable toxicity in these experiments, probably owing to the low solubility constant of PbSe (Kₛₚ=3×10⁻²⁸) and the relatively short exposure time. To provide a completely nontoxic solution, inside-coated glass microtips were also developed as photoelectrodes. This led to a nanostructured photoelectrode encapsulated by a thin glass layer. The inside-coated photoelectrode was also tested for another more fundamental reason. The question arose regarding whether the neuron stimulation results from a chemical electron-transfer process, which would require direct contact through the solution phase, or if it results from an electric field effect. This question is relevant in order to understand the underlying phototriggering process. If inside-coated photoelectrodes that were completely enclosed by glass would produce a phototrigger for nearby neurons, evidence for a local electric field effect would be obtained. Indeed, the inside-coated microtips (FIG. 3c, right), with which there is minimal chemical contact between the nanostructured semiconductor thin film and the neuron cells, show the same effect in phototriggering neurons as the outsidecoated photoelectrodes. The formation of a photoinduced electric field was tested by electrical potential measurements near glass microtips coated with PbSe nanoparticles (FIG. 4); in these measurements, two recording tungsten metal electrodes (Rec) were placed near the stimulating microelectrode (Stim), and the voltage differential was measured. The results showed that there is a potential formed between the two recording electrodes when the stimulating photoelectrode was irradiated with light. The photoinduced potential and lack of measurable photocurrents together suggest that it is a light-induced field effect from the nanoparticle film, which causes the stimulation of neurons. The same setup was also used to measure the photoinduced potential on glass microtips without PbSe nanoparticle coating, which failed to induce a detectable photopotential. The results suggest that the phototriggering could be activated by a photoinduced electric field caused by trapped charge carriers in the PbSe nanoparticle film. Of course, the possibility of small currents, which would originate from photogenerated surface charges, cannot be excluded completely.

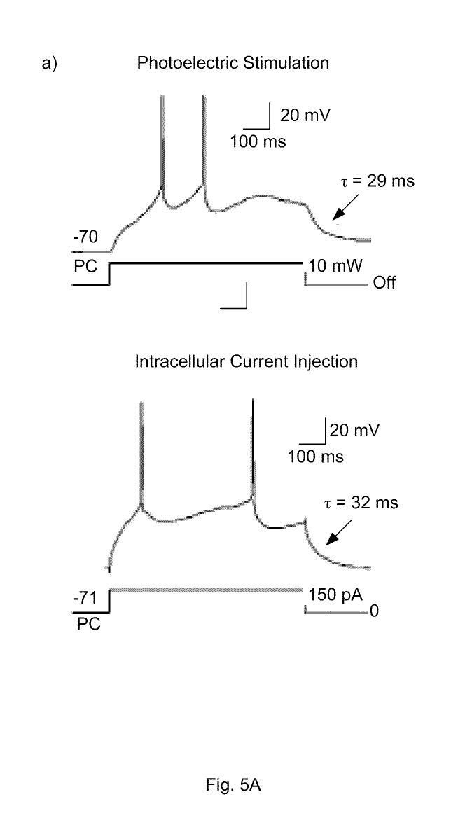

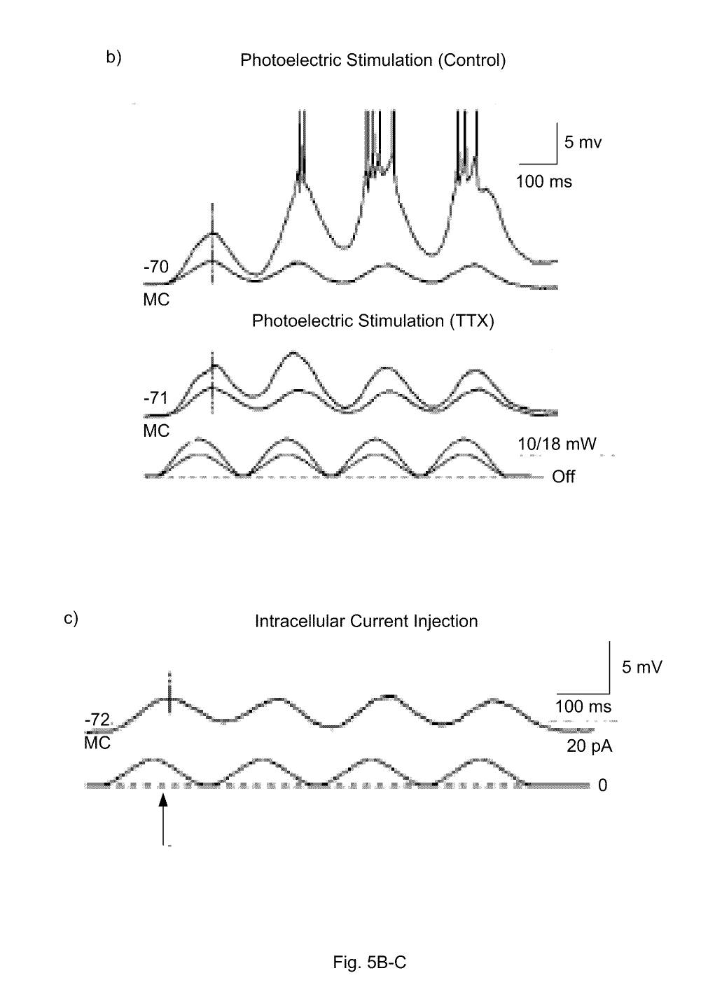

The neural stimulation could be modulated in time and intensity. In FIG. 5a a comparison of photoelectric activation (top; 830 nm pulsed beam; 10 m W average illumination measured at the objective focal point) and depolarization of the same neocortical L2/3 pyramidal cell with the direct current-injection step (bottom) is shown. Both stimulation methods depolarized the neuron to threshold and triggered two action potentials. The neural membrane potential recovered with the same time constant (about 30 ms) following photoexcitation and direct-current-injection steps, suggesting that the kinetics of both responses were governed by the neuronal membrane time constant.

Rapid laser scanning over the nanostructured stimulation photoelectrode (PE Stim) effectively depolarized both neocortical pyramidal cells (n=11) and olfactory bulb neurons (n=11). Neurons could be activated repetitively without apparent damage such as long-lasting changes in resting membrane potential, input resistance, and action potential amplitude. Focal illumination of a similar area equidistant to the recorded cell but not covering the stimulation electrode (PE Stim) failed to trigger intracellular depolarization (data not shown). No responses were detected when the laser beam was scanned over an uncoated patch pipette within 10-20 μm of the recorded neuron at similar illumination intensities (5-20 mW).

Photoelectric neuronal activation reflects predominately passive depolarization and can be elicited in the presence of blockers of fast synaptic transmission (5 μm NBQX, 25 μm D-APV, 10 μm gabazine; obtained from Sigma; FIG. 5c, top). Neuronal responses could be patterned by applying arbitrary functions to the Pockels cell light intensity control, such as the responses to sine wave stimuli shown in FIG. 5 c. Photoelectric responses were graded with mean illumination intensity and subthreshold responses were unaffected by the Na⁺ channel blocker tetrodotoxin (TTX, 1 μm; FIG. 5b, down), suggesting that light-triggered responses were mediated directly, and not through activation of other nearby cells.

Responses to sinusoidal illumination intensity modulation showed a similar phase lag to responses evoked by current waveforms injected intracellularly in the same neurons (28.6±2.5 vs. 29.8±0.7 ms, respectively; mean±SEM; p>0.5; FIG. 5c, bottom trace), implying that the kinetics of both responses were governed by passive membrane properties.

Focal illumination of stimulation electrodes (PE Stim) coated on their inside surfaces also depolarized neocortical and olfactory bulb neurons (n=4; 830 nm illumination; 5-20 mW average intensity), supporting that electrical fields, and not currents, mediate this effect. These results suggest that nanoparticle-coated micropipettes can effectively activate neurons in intact brain tissue in response to light pulses. This stimulation technique offers a simpler alternative to genetically encoded light switches or bath-applied caged compounds for neuronal stimulation. Nanoparticle-based photoelectric neuronal activation will likely prove useful clinically to activate brain regions and damaged nerves.

From the above description of the invention, those skilled in the art will perceive improvements, changes and modifications Such improvements, changes and modifications are within the skill of the art and are intended to be covered by the appended claims. All publications, patents, and patent applications cited in the present application are herein incorporated by reference in their entirety.

Having described the invention, the following is claimed:

1. A photoelectric stimulating electrode for photoelectric activation of a neuron, comprising:
a plurality of semiconductor nanoparticles adapted to be positioned proximate the neuron, the semiconductor nanoparticles upon excitation with light of a first wave length generating an electric field and/or current effective to stimulate the neuron.

2. The electrode of claim 1, the semiconductor nanoparticles generating an electric field and/or current upon excitation with visible and/or near-infrared light effective to stimulate the neuron.

3. The electrode of claim 1, the semiconductor nanoparticles generating an electric field and/or current upon excitation of light having a wavelength of about 650 nm to about 2200 nm.

4. The electrode of claim 1, the semiconductor nanoparticles being provided in and/or on a biocompatible substrate.

5. The electrode of claim 4, the substrate having an area that is adapted to be positioned proximate a plurality of neurons, the semiconductor nanoparticles being provided in an array within the area.

6. The electrode of claim 4, the substrate being substantially transparent light effective to excite the semiconductor nanoparticles.

7. The electrode of claim 1, the semiconductor nanoparticles comprising semiconductor material having a band gap less than about 2 eV at a temperature of 300K.

8. The electrode of claim 7, the semiconductor nanoparticles comprising PbSe.

9. An apparatus for photoelectric activation of a neuron, comprising
a plurality semiconductor nanoparticles adapted to be positioned proximate the neuron, the semiconductor nanoparticles generating an electric field and/or current effective to stimulate the neuron upon excitation with light of a first wavelength; and
a light generating mean for generating light having the first wavelength effective to excite the semiconductor nanoparticles to generate the electric field and/or current.

10. The apparatus of claim 9, further comprising a substrate, the semiconductor nanoparticles being provided in and/or on the surface of substrate.

11. The apparatus of claim 9, substrate being biocompatible with the neurons being stimulated.

12. The apparatus of claim 9, the semiconductor nanoparticles being provided in a film that coats at least portion of a surface of the substrate.

13. The apparatus of claim 9, the semiconductor nanoparticles generating an electric field and/or current upon excitation with visible and/or near-infrared light effective to stimulate the neuron.

14. The apparatus of claim 9, the semiconductor nanoparticles generating an electric field and/or current upon excitation of light having a wavelength of about 650 nm to about 2200 nm.

15. The apparatus of claim 9, the semiconductor nanoparticles comprising semiconductor material having a band gap less than about 2 eV at a temperature of 300K.

16. The apparatus of claim 9, the semiconductor nanoparticles comprising PbSe.

17. A method of stimulating a neuron, the method comprising:
positioning a plurality semiconductor nanoparticles proximate the neuron, the semiconductor nanoparticles generating an electric field and/or current effective to stimulate the neuron upon excitation with light of a first wavelength; and
exciting the plurality of semiconductor nanoparticles with light having the first wavelength to generate the electric field and/or current.

18. The method of claim 17, the semiconductor nanoparticles being provided on a surface of a substrate.

19. The method of claim 17, the substrate being biocompatible with the neurons being stimulated.

20. The method of claim 17, the semiconductor nanoparticles being provided in a film that coats at least portion of a surface of a substrate.

21. The method of claim 17, the semiconductor nanoparticles generating an electric field and/or current upon excitation of light having a wavelength of about 650 nm to about 2200 nm.

22. The method of claim 17, the semiconductor nanoparticles comprising semiconductor material having a band gap less than about 2 eV at a temperature of 300K.

23. The method of claim 22, the semiconductor nanoparticles comprising PbSe.
